(19) United States
(12) Patent Application Publication
(10) Pub. No.: US 2017/0021083 A1
(43) Pub. Date: Jan. 26, 2017

(54) APHERESIS SYSTEM

(71) Applicant: Parker-Hannifin Corporation, Cleveland, OH (US)

(72) Inventors: Paul Gray, Dunstable (GB); Michael Collinson, Camarillo, CA (US)

(21) Appl. No.: 15/122,007

(22) PCT Filed: Feb. 25, 2015
(86) PCT No.: PCT/US2015/017434

§ 371 Date: Aug. 26, 2016

Related U.S. Application Data
(60) Provisional application No. 61/946,172, filed on Feb. 28, 2014.

(51) Int. Cl. A61M 1/34; G01L 19/00; G01L 19/14; A61M 1/36

(52) U.S. Cl. CPC A61M 1/3403; A61M 1/3496; A61M 1/3639; A61M 1/367; A61M 1/3638; A61M 1/342; A61M 1/3413; A61M 1/3626; G01L 19/0023; G01L 19/0038; G01L 19/0084; G01L 19/147; A61M 2205/3331; A61M 2230/20

(57) ABSTRACT

A therapeutic apheresis system including a tube set and a panel is presented. The tube set includes an in-line pressure sensor in fluid connection with tubing. The panel includes apertures that are aligned with electrical connectors through which a rigid plug portion of an in-line pressure sensors extends and makes electrical connection with at least one electrical connector.

### FIELD OF INVENTION

The present disclosure relates generally to immunoadsorption therapy, and more particularly to apheresis systems.

### BACKGROUND

Therapeutic apheresis systems are used to remove a component of the blood which contributes to a disease state. Such systems utilize tube sets to remove blood from a patient, deliver the blood to filters, transport separated blood components, and deliver a portion of the blood back to the patient.

Monitoring the fluid pressure in different tubing portions of a therapeutic apheresis systems is necessary to ensure accurate functioning of the system and safe return of blood components to the patient. It is also important to minimize the complexity of a tube set and its connection to the system to minimize the likelihood of incorrectly connecting the tube set to the system.

Current therapeutic apheresis systems use a remote pressure sensor to monitor the pressure in different portions of the tube set. The remote pressure sensor is connected via a long flexible tube to each portion of the tube set where pressure is monitored. The long flexible tubes may introduce errors into the pressure measurements due to, e.g., heating or cooling of air in the tube. The additional long flexible tubes (one for each measurement point) may also complicate connecting the tube set to the therapeutic apheresis system. That is, the long flexible tubes increase the number of tube portions in the tube set, increasing the chances that a user will incorrectly connect the tube set to the therapeutic apheresis system.

### SUMMARY

The present disclosure provides a therapeutic apheresis system including a panel having apertures that are aligned with electrical connectors through which a rigid plug portion of an in-line pressure sensor extends and makes electrical connection with at least one of the electrical connectors.

According to one aspect of the disclosure, there is provided an in-line pressure sensor including a housing and a pressure transducer. The housing includes a fluid flow passage, a window open to the fluid flow passage, and a rigid plug portion fixed in relation to the housing and extending in a direction transverse to the fluid flow passage. The rigid plug portion includes at least one electrical contact for mating with an external component. The pressure transducer includes a sensing region in pressure communication with the passage and a wiring contact in electrical connection with the at least one electrical contact.

Alternatively or additionally, a tube set includes tubing and at least one in-line pressure sensor. The at least one in-line pressure sensor is fluidly connected with a portion of the tubing.

Alternatively or additionally, a therapeutic apheresis system includes the in-line pressure sensor, a panel, a printed circuit board (PCB) mounted adjacent a back side of the panel and including at least one electrical connector. The panel has an aperture that is aligned with the electrical connector and through which the rigid plug portion of the in-line pressure sensor extends and makes electrical connection with the electrical connector.

According to another aspect of the disclosure, a therapeutic apheresis system includes an in-line pressure sensor, a panel, and a printed circuit board. The in-line pressure sensor includes a fluid flow passage at a first end of the in-line pressure sensor and at least one electrical contact at a second end of the in-line pressure sensor. The printed circuit board (PCB) mounted adjacent a back side of the panel and includes at least one electrical connector. The panel has an aperture that extends from a front side of the panel to a back side of the panel and is aligned with the electrical connector, the second end of the in-line pressure sensor extends through the aperture and making electrical contact with the electrical connector.

According to a further aspect of the disclosure, a therapeutic apheresis system includes a covering having a panel, at least one peristaltic pump supported on the panel, a display on the panel, a primary filter for receiving blood and separating a cellular component of the blood from a plasma component of the blood, at least one receptacle for receiving a portion of a tube set that connects the pump to the primary filter, and electrical circuitry contained within the housing to control operation of the at least one peristaltic pump.

Alternatively or additionally, the therapeutic apheresis system additionally includes a tube set including tubing and the in-line pressure sensor. The at least one in-line pressure sensor is fluidly connected with a portion of the tubing.

Alternatively or additionally, the tube set includes a plurality of in-line pressure sensors. The plurality of in-line pressure sensors include an arterial pressure sensor, a pre-filter pressure sensor, an ultrafiltrate pressure sensor, a post-column pressure sensor, and a venous pressure sensor. The tubing includes multiple portions including arterial tubing, cellular components venous tubing, plasma tubing, post-column tubing, and venous tubing. The arterial tubing has an input end and an output end and includes the arterial pressure sensor and the pre-filter pressure sensor, the arterial pressure sensor located closer to the input end of the arterial tubing than the pre-filter pressure sensor. The cellular components venous tubing has an input end and an output end. The plasma tubing has an input end and an output end and includes the ultrafiltrate pressure sensor. The post-column tubing has an input end and an output end and includes the post-column pressure sensor. The venous tubing has an input end and an output end and includes the venous pressure sensor.

Alternatively or additionally, the tube set further includes a bubble trap filter having a plasma input, a cellular content input, and an output. The bubble trap filter is configured to receive cellular components of blood at the cellular content input, receive a plasma component of the blood at the plasma input, combine the plasma component of the blood and the cellular components of the blood into a blood mixture, and output the blood mixture at the output of the bubble trap filter. The plasma input of the bubble trap filter is fluidly connected with the output end of the post-column tubing. The cellular content input of the bubble trap filter is fluidly connected with the output end of the cellular components venous tubing. The output of the bubble trap filter is fluidly connected to the input end of the venous tubing.

Alternatively or additionally, the tube set further includes a primary filter and a column. The primary filter has an arterial input, a plasma output, and a cellular components venous output. The primary filter is configured to receive blood at the arterial input, separate a plasma component of the blood from cellular components of the blood, output the cellular components of the blood at the cellular components venous output of the primary filter, and output the plasma component of the blood at the plasma output of the primary filter. The arterial input of the primary filter is fluidly connected to the output end of the arterial tubing. The cellular components venous output of the primary filter is fluidly connected to the input end of the cellular components venous tubing. The plasma output of the primary filter is fluidly connected to the input end of the plasma tubing. The column has an input and an output. The column is configured to receive the plasma of the blood at the input of the column, remove unwanted constituents from the plasma of the blood, and output the plasma of the blood at the output of the column. The input of the column is fluidly connected with the output end of the plasma tubing. The output of the column is fluidly connected with the input end of the post-column tubing.

Alternatively or additionally, the panel includes a receptacle for receiving a length of the tubing.

Alternatively or additionally, the panel includes a plurality of receptacles for holding respective portions of the tube set.

Alternatively or additionally, the receptacle includes a clip or a channel.

Alternatively or additionally, the therapeutic apheresis system includes a printed circuit board (PCB) mounted adjacent a back side of the panel and includes at least one electrical connector. The panel has an aperture that extends from a front side of the panel to a back side of the panel and is aligned with the electrical connector.

Alternatively or additionally, the aperture includes a retainer for holding the rigid plug portion in engagement with the electrical connector when the plug portion is engaged with the electrical connector.

Alternatively or additionally, the at least one electrical connector is electrically connected to an amplifier disposed on the PCB.

Alternatively or additionally, the therapeutic apheresis system further includes an arterial pump applying force to the arterial tubing at a location between the arterial pressure sensor and the pre-filter pressure sensor such that the contents of the arterial tubing are propelled towards the output end of the arterial tubing.

Alternatively or additionally, the therapeutic apheresis system further includes a substitution pump and substitution tubing having an input end and an output end. The input end of the substitution tubing is fluidly connected to a substitution reservoir. The substitution pump applies force to the substitution tubing and propels the contents of the substitution tubing towards the output end of the substitution tubing. The output end of the substitution tubing is fluidly coupled to the arterial tubing nearer the input end of the arterial tubing than the arterial pressure sensor.

Alternatively or additionally, the substitution tubing includes a substitution non-return valve located nearer the output end of the substitution tubing than the substitution pump.

Alternatively or additionally, the substitution pump is a peristaltic pump.

Alternatively or additionally, the arterial pump is a peristaltic pump.

Alternatively or additionally, the therapeutic apheresis system further includes a plasma pump applying force to the post-column tubing at a location between the post-column pressure sensor and the output end of the post-column tubing such that the contents of the post-column tubing are propelled towards the output end of the post-column tubing.

Alternatively or additionally, the plasma pump is a peristaltic pump.

Alternatively or additionally, the post-column tubing additionally includes a particle trap filter located between the post-column pressure sensor and the plasma pump.

Alternatively or additionally, the therapeutic apheresis system further includes a non-return valve. The non-return valve is located nearer the input end of the plasma tubing than the ultrafiltrate pressure sensor.

Alternatively or additionally, the therapeutic apheresis system further includes a hemoglobin detector located nearer the output end of the plasma tubing than the ultrafiltrate pressure sensor.

Alternatively or additionally, the tube set further includes waste tubing having an input end, an output end, and a waste clamp. The input end of the waste tubing is fluidly connected to the plasma tubing nearer the output end of the plasma tubing than the hemoglobin detector. The waste clamp is configured to block fluid flow through the waste tubing when activated and the waste clamp is located between the input end of the waste tubing and the output end of the waste tubing. The output end of the waste tubing is fluidly connected with a waste reservoir.

Alternatively or additionally, the venous tubing additionally includes a bubble detector located between the venous pressure sensor and the output end of the venous tubing.

Alternatively or additionally, the venous tubing further includes a venous clamp located between the bubble detector and the output end of the venous tubing, the venous clamp is configured to block fluid flow through the venous tubing when activated.

The foregoing and other features of the disclosure are hereinafter fully described and particularly pointed out in the claims, the following description and annexed drawings setting forth in detail certain illustrative embodiments of the disclosure, these embodiments being indicative, however, of but a few of the various ways in which the principles of the disclosure may be employed.

Features that are described and/or illustrated with respect to one embodiment may be used in the same way or in a similar way in one or more other embodiments and/or in combination with or instead of the features of the other embodiments.

### BRIEF DESCRIPTION OF THE DRAWINGS

FIG. 1A is a perspective view of an exemplary therapeutic apheresis system including a tube set.

FIG. 1B is a perspective view of the therapeutic apheresis system of FIG. 1 without a tube set.

FIGS. 2A and 2B are cross-sectional views of a portion of the therapeutic apheresis system of FIG. 1A.

FIG. 3A is a perspective view of an in-line pressure sensor.

FIG. 3B is a perspective view of the in-line pressure sensor of FIG. 3A with a portion of a housing removed.

FIG. 3C is a perspective view of the in-line pressure sensor of FIG. 3B with a pressure transducer and electrical contact removed.

FIG. 3D is a perspective view of the pressure transducer of FIG. 3C.

FIGS. 4A and 4B are perspective views of a tube set.

### DETAILED DESCRIPTION

The present disclosure provides a therapeutic apheresis system including a tube set and a panel. The tube set includes an in-line pressure sensor in fluid connection with tubing. The panel includes apertures that are aligned with electrical connectors through which a rigid plug portion of an in-line pressure sensors extends and makes electrical connection with at least one electrical connector.

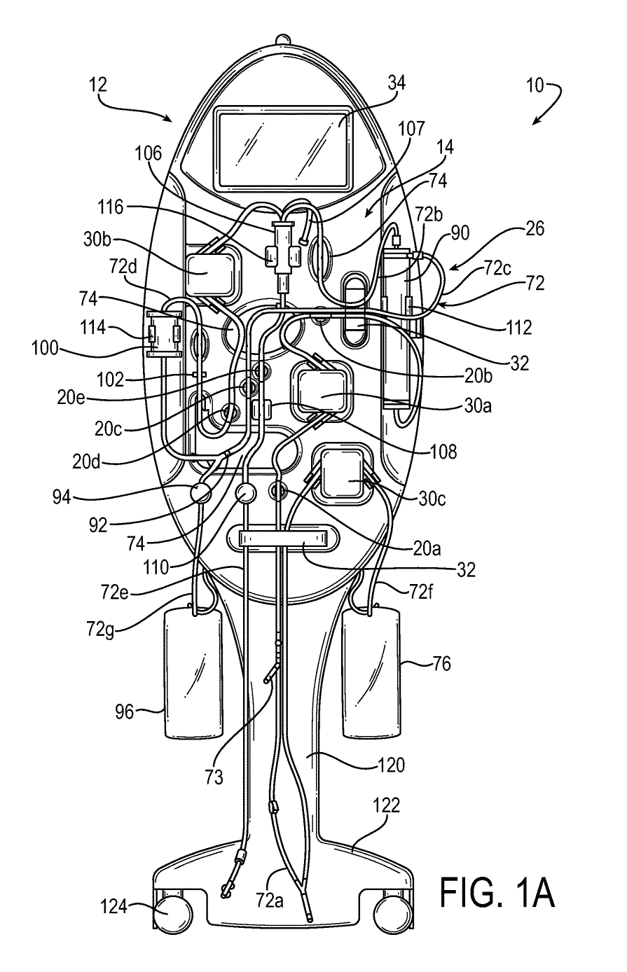

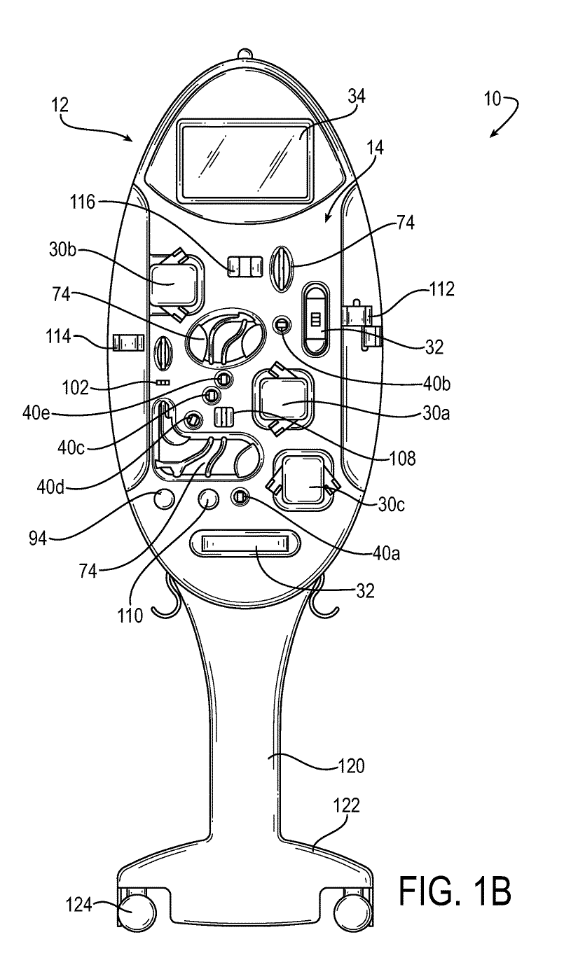

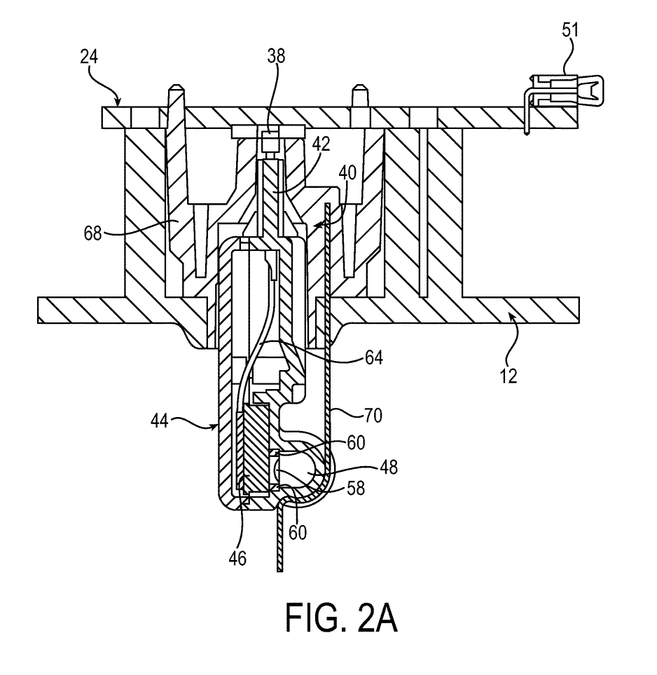

Turning initially to FIGS. 1A and 1B, an exemplary therapeutic apheresis system 10 is shown. The therapeutic apheresis system 10 includes a covering 12 having a panel 14, at least one in-line pressure sensor 20, and a printed circuit board (PCB) 24 (FIG. 2A). As shown, the at least one in-line pressure sensor 20 may be included as part of a tube set 26. The therapeutic apheresis system 10 may also include at least one pump 30 supported on the panel 14 and at least one receptacle for receiving a portion of the tube set 26.

The therapeutic apheresis system 10 may also include electrical circuitry (not shown). The electrical circuitry may be contained within the covering 12 and used to control operation of the at least one pump 30. The electrical circuitry may comprise, e.g., a controller or processor. A user may interact with and issue commands to the electrical circuitry through a display 34 included on the panel 14. The electrical circuitry may receive data from sensors included in the therapeutic apheresis system 10. For example, the electrical circuitry may control the at least one pump 30 based on received pressure data from the at least one in-line pressure sensor 20 and/or user entered commands.

As will be understood by one of ordinary skill in the art, the electrical circuitry may have various implementations. For example, the electrical circuitry may include any suitable device, such as a programmable circuit, integrated circuit, memory and I/O circuits, an application specific integrated circuit, microcontroller, complex programmable logic device, other programmable circuits, or the like. The electrical circuitry may also include a non-transitory computer readable medium, such as random access memory (RAM), a read-only memory (ROM), an erasable programmable read-only memory (EPROM or Flash memory), or any other suitable medium. Instructions for controlling operation of the therapeutic apheresis system 10 may be stored in the non-transitory computer readable medium and executed by the electrical circuitry. The electrical circuitry may be communicatively coupled to a computer readable medium through a system bus, mother board, or using any other suitable structure known in the art.

The in-line pressure sensor 20 interfaces with the therapeutic apheresis system 10 in order to provide the therapeutic apheresis system 10 with a measured pressure of a fluid in a fluid flow passage of the in-line pressure sensor 20. As shown in FIGS. 2A and 2B, the in-line pressure sensor 20 interfaces with the therapeutic apheresis system 10 via the PCB 24. The PCB 24 is mounted adjacent a back side 36 of the panel 14 and includes at least one electrical connector 38. The panel 14 has an aperture 40 that is aligned with the electrical connector 38. A rigid plug portion 42 of the in-line pressure sensor 20 extends through the aperture 40 and makes electrical connection with the electrical connector 38.

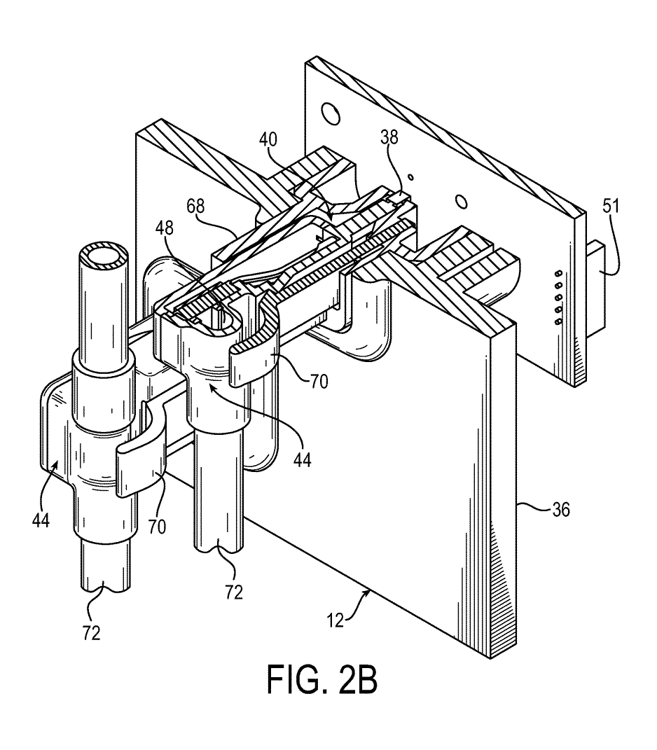

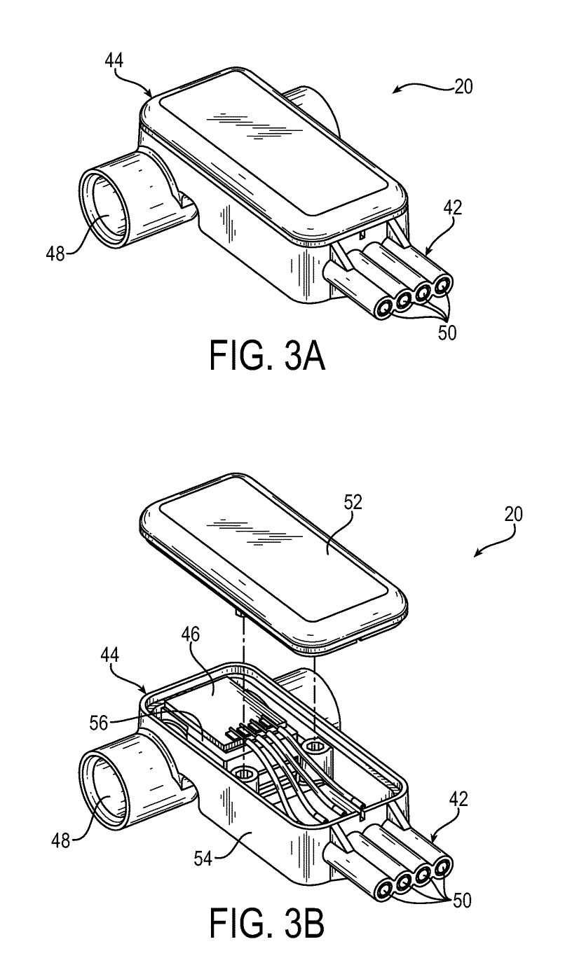

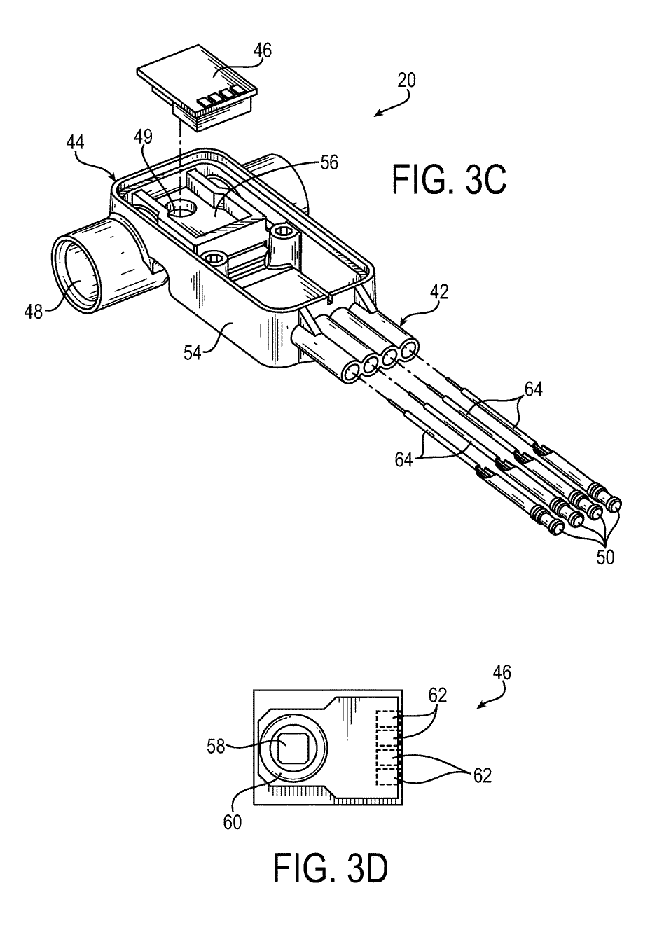

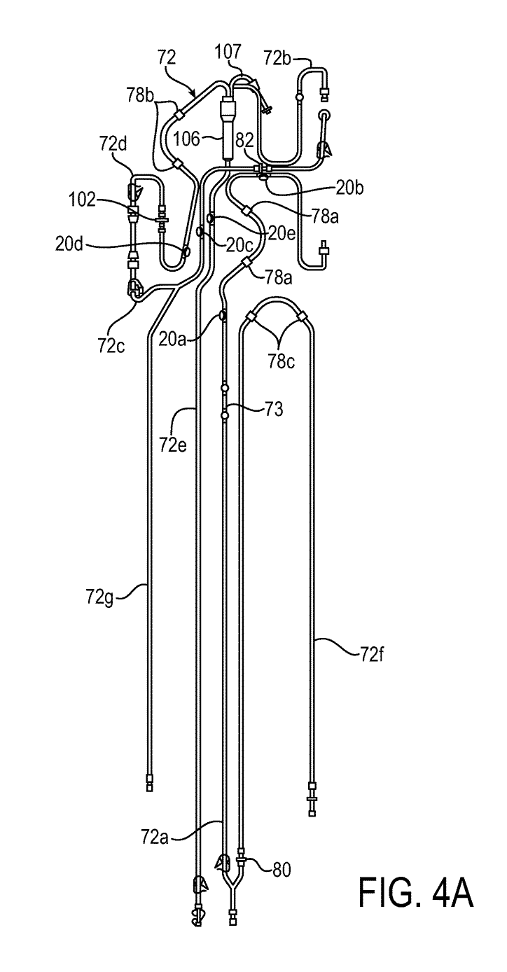

As shown in FIGS. 2A and 2B, the aperture 40 may include a pressure sensor receiver 68 configured to receive and position the rigid plug portion 42 of the in-line pressure sensor 20 such that an electrical contact 50 (FIG. 3B) of the in-line pressure sensor 20 makes connection with the electrical connector 38. That is, the pressure sensor receiver 68 may be shaped such that, as the rigid plug portion 42 is inserted into the aperture 40, the rigid plug portion 42 is directed towards the electrical connector 38. An interior of the pressure sensor receiver 68 may narrow at positions closer to the electrical connector 38.

The pressure sensor receiver 68 may be made of any suitable material. For example, the pressure sensor receiver 68 may be made of a plastic material.

The aperture 40 may also include a retainer 70 for holding the rigid plug portion 42 in engagement with the electrical connector 38 when the plug portion 42 is engaged with the electrical connector 38. The retainer 70 may comprise a structure having a shape that conforms to an outer surface of a housing 44 of the in-line pressure sensor 20. The retainer 70 may be deflected out of position as the in-line pressure sensor 20 is inserted into the aperture 40. When the in-line pressure sensor 20 is inserted into the aperture 40 such that the electrical connector 38 of the PCB 24 is engaged with the electrical contact 50 of the in-line pressure sensor, the retainer 70 may return to its initial position as shown in FIG. 2A. In this position, the retainer 70 may need to be deformed out of its current position in order to remove the in-line pressure sensor 20 from the aperture 40.

The retainer 70 may be made from any suitable material capable of deflection from and returning to the initial position. For example, the retainer 70 may be made from a metal such as steel, aluminum, or any other suitable substance as will be understood by one of ordinary skill in the art. As shown in FIGS. 2A and 2B, the retainer 70 may be attached at one end to the pressure sensor receiver 68.

The PCB 24 may include an amplifier disposed on the PCB 24. The at least one electrical connector 38 of the PCB 24 may be electrically connected to the amplifier. In one embodiment, the amplifier may be positioned in close proximity to the electrical contact 50. The PCB 24 may also include an output connector 51 configured to output the electrical signals received by the electrical connector 38 of the PCB 24. In this embodiment, the amplifier may be located adjacent to the PCB 24 and connected electrically and/or physically to the output connector of the PCB 24. By positioning the amplifier in close proximity to the electrical connector 38, the therapeutic apheresis system 10 reduces the introduction of noise into the measured pressure data.

As shown in FIGS. 3A-3D, the in-line pressure sensor 20 may include a housing 44 and a pressure transducer 46. The housing 44 includes the fluid flow passage 48 located at a first end of the in-line pressure sensor 20, a window 49 open to the fluid flow passage 48, and the rigid plug portion 42. The rigid plug portion 42 is fixed in relation to the housing 44 and extends in a direction transverse to the fluid flow passage 48. The rigid plug portion 42 includes at least one electrical contact 50 for meeting with an external component. The electrical contact 50 is located at a second end of the in-line pressure sensor of 20. It is the second end of the in-line pressure sensor 20 that extends through the aperture 40 and makes electrical contact with the electrical connector 38. The fluid flow passage 48 may be located at an opposite end of the in-line pressure sensor as the electrical contact 50. The electrical contact 50 may comprise Harwin pins or any suitable element capable of transferring an electrical signal.

As shown in FIG. 3B, the housing 44 may include a lid 52 and a main body 54. The lid 52 and main body 54 may fit together in order to maintain the position of the pressure transducer 46 relative to the window 49. The main body 54 may include a pressure transducer seat 56 configured to receive the pressure transducer 46. The pressure transducer seat 56 is configured to receive the pressure transducer 46 such that a sensing elements 58 of the pressure transducer 46 is aligned with the window 49. When the pressure transducer 46 is positioned within the housing 44, the sensing elements 58 and the window 49 are aligned such that the sensing element 58 is in pressure communication with the fluid flow passage 48.

The pressure transducer 46 may include a seal 60 that surrounds a perimeter of the sensing elements 58. When the pressure transducer is positioned in the pressure transducer seat 56, the seal 60 may lie against the pressure transducer seat 56 surrounding the outer perimeter of the window 49. In this position, fluid flowing within the fluid flow passage 48 may be prevented from entering the interior of the housing 44. In another embodiment, the seal 60 has a diameter smaller than a diameter of the window 49, such that the seal 60 fits within the window 49. Again, in this position, the seal 60 prevents fluid from entering the interior of the housing 44.

As will be understood by one of ordinary skill in the art, the seal 60 may take any form suitable to prevent leakage of fluid within the fluid flow passage 48 into an interior of the housing 44. Rather than exposing the sensing elements 58 directly to the fluid within the fluid flow passage 48, a diaphragm or thin sheet of material may be positioned between the sensing elements 58 and the fluid flow passage 48. In this embodiment, the diaphragm may be configured such that the sensing element 58 is in pressure communication with the fluid flow passage 48, but is not in direct contact with the fluid flow passage 48.

The pressure transducer 46 may additionally include a vent (not shown) to determine the atmospheric pressure.

As used herein, pressure communication signifies that the sensing element 58 is capable of measuring the pressure of a fluid. Pressure communication may signify direct communication between a fluid and a sensor or the presence of a material between the fluid and a sensor.

As will be understood by one of ordinary skill in the art, the housing 44 may be made of any suitable material capable of providing a housing 44 to the pressure transducer 46. The housing may be made from medically approved materials that are sterilized. For example, the housing 44 may be made of a rigid plastic. For example, the housing may be injection molded. By forming the housing 44 of a rigid plastic, connection of the tube set 26 containing the at least one in-line pressure sensor 20 may be simplified. That is, reducing the use of non-rigid wires to connect the in-line pressure sensor 20 to the therapeutic apheresis system 10 reduces the presence of loose wires in the system that may interfere with accurate connection of the tube set 26 to the therapeutic apheresis system 10.

The pressure transducer may include at least one electrical contact point 62. The at least one electrical contact 50 of the in-line pressure transducer 20 may be connected to the at least one electrical contact point 62 by an intermediate wire 64. Each electrical contact point 62 may be connected to a single electrical contact 50 by a single intermediate wire 64. The pressure transducer 46 may receive power and/or output measured pressure data via the at least one electrical contact 62.

As will be understood by one of ordinary skill in the art, the pressure transducer 46 may comprise any suitable pressure sensor capable of measuring a pressure of a fluid and/or air located within the fluid flow passage 48. For example, the pressure transducer 46 may comprise a wet sensor.

Turning to FIG. 1A, the at least one in-line pressure sensor 20 may be included as part of a tube set 26. The tube set 26 may include tubing 72 and the at least one in-line pressure sensor 20. The at least one in-line pressure sensor 20 is fluidly connected with a portion of the tubing 72. The panel 14 of the therapeutic apheresis system may include a receptacle 74 for receiving a length of the tubing 72. For example, as shown in FIGS. 1A and 1B, the panel 14 includes a plurality of receptacles 74 for holding respective portions of the tube set 26. The receptacles 74 may include a raised portion of the panel 14 containing channels with indentations configured to hold a portion of tubing inserted into the channel. The receptacles 74 may improve connection of the tube set 26 to the therapeutic apheresis system 10. For example, the receptacles 74 maintain the position of different portions of the tubing 26 near the panel 14, reducing tangling of the tubing 26 during installation. The receptacles 74 also aid in placement of the tubing 26 during connection to the therapeutic apheresis system 10, reducing user error.

The panel 14 and the receptacles 74 may be formed from a rigid plastic material. As will be understood by one of ordinary skill in the art, the panel 14 may be formed of any suitable material. The receptacles 74 may be composed of the same material as the panel 14. In one embodiment, the receptacles 74 are formed as protrusions from the panel 14. The protruding receptacles 74 include channels (i.e. indentations) in the receptacles 74 through which portions of the tubing 26 may be inserted and retained. In an alternative embodiment, the receptacles may be formed from a different material than the panel 14. For example, the receptacles may be formed from a rubber or similar material having a higher coefficient of friction than the panel 14. In this example, the receptacles are formed as protrusions from the panel 14 including channels for holding portions of the tubing 26. The higher coefficient of friction of the receptacles 74 in this example may provide improved holding of the tubing 26. Additionally, forming the receptacles from a rubberlike material may enhance the user's ability to insert the tubing 26 into the receptacle 74.

As will be understood by one of ordinary skill in the art, the receptacles 74 may include a clip, channel, or any other suitable structure for holding or maintaining the position of portions of the tube set 26.

In one embodiment, the tube set 26 includes a plurality of in-line pressure sensors 20a-e. The plurality of in-line pressure sensors include an arterial pressure sensor 20a, a pre-filter pressure sensor 20b, an ultrafiltrate pressure sensor 20c, a post-column pressure sensor 20d, and a venous pressure sensor 20e.

The tubing 72 of the tube set 26 may include multiple portions, including an arterial tubing 72a, a cellular components venous tubing 72b, a plasma tubing 72c, a post-column tubing 72d, and a venous tubing 72e. The arterial tubing 72a may have an input end and an output end and include the arterial pressure sensor 20a and the pre-filter pressure sensor 20b. The cellular components venous tubing 72b may have an input end and an output end. The plasma tubing 72c may include an input end and an output end and include the ultrafiltrate pressure sensor 20c. The post-column tubing 72d may have an input end and an output end and include the post-column pressure sensor 20d. The venous tubing 72e may have an input end and an output end and include the venous pressure sensor 20e.

As will be understood by one of ordinary skill in the art, the tubing 72 may be connected to the in-line pressure sensors 20 using any suitable means. For example, each in-line pressure sensor 20 may be bonded to a given portion of tubing 72.

The tubing 72 may be made from any suitable material and have any suitable diameter for transporting biological fluids such as blood or blood components. For example, the tubing 72 may be made from a biologically compatible plastic that does not illicit an immune response from the blood. That is, the plastic does not interact with or cause an interaction from the blood. The tubing 72 may also have an inner diameter of 4.1 mm and an outer diameter of 6.8 mm.

A patient may be connected to the input end of the arterial tubing 72a such that the patient's blood is drawn into the arterial tubing 72a. The pressure of the patient's blood in the tubing 72a may be monitored by the arterial pressure sensor 20a. The patient's blood may be propelled through the arterial tubing 72a by an arterial pump 30a. The pressure exerted on the blood in the arterial tubing 72a by the arterial pump 30a may be monitored by the pre-filter pressure sensor 20b. That is, the therapeutic apheresis system 10 may include the arterial pump 30a located at a point along the arterial tubing 72a between the arterial pressure sensor 20a and the pre-filter pressure sensor 20b. The arterial pump 30a applies force to the arterial tubing 72a such that the contents of the arterial tubing 72a are propelled towards the output end of the arterial tubing 72a. In this way a portion of the tube set 26 connects the pump 30 to the primary filter 90.

The electrical circuitry may control the arterial pump 30a such that the pressure measured by the pre-filter pressure sensor 20a is maintained within a predefined arterial pressure range. The predefined arterial pressure range may be defined by a user or may be a default setting of the system 10.

The arterial tubing 72a may include two markers 78a indicating the position for connecting the arterial pump 30a to the arterial tubing 72a. That is, the arterial tubing 72a may be connected to the therapeutic apheresis system 10 such that the arterial pump 30a interacts with the arterial tubing 72a between the two markers 78a. The markers 78a may also function to maintain the position of the arterial tubing 72a relative to the substitution pump 30a.

The arterial tubing 72a may additionally include a side branch tubing 73. The side branch tubing 73 may be configured for adding additional components to the arterial tubing 72a. For example, the side branch tubing 73 may be used to add drugs or Leparin to the arterial tubing 72a.

The therapeutic apheresis system may also include a substitution pump 30c. In this embodiment, the tube set 26 may also include a substitution tubing 72f having an input end and an output end. The input end of the substitution tubing 72f may be fluidly connected to a substitution reservoir 76. The substitution reservoir 76 may store an anticoagulant or other material that is mixed with the patient's blood as it enters the arterial tubing 72f. The anticoagulant reduces coagulation of the blood in the tube set 26. The substitution pump 30c applies force to the substitution tubing 72f and propels the contents of the substitution tubing 72f towards the output end of the substitution tubing 72f. The output end of the substitution tubing 72f is fluidly coupled to the arterial tubing 72a nearer the input end of the arterial tubing 72a than the arterial pressure sensor 20a.

The substitution tubing 72f may include two markers 78c indicating the position for connecting the substitution pump 30c to the substitution tubing 72f. That is, the substitution tubing 72f may be connected to the therapeutic apheresis system 10 such that the substitution pump 30c interacts with the substitution tubing 72f between the two markers 78c. The markers 78c may also function to maintain the position of the substitution tubing 72f relative to the substitution pump 30c.

The electrical circuitry may also be configured to control the substitution pump 30c to transport anticoagulant or other material through the substitution tubing 72f at a predefined substitution rate. The predefined substitution rate may be adjusted and/or inputted by a user using the display 34. The electrical circuitry may determine the amount of anticoagulant or other material transported through the substitution tubing 72f using a load cell measuring the weight of anticoagulant or other material remaining in the substitution reservoir 76.

The substitution tubing 72f may include a substitution non-return valve 80 located nearer the output end of the substitution tubing 72f than the substitution pump 30c. The substitution non-return valve 80 functions to ensure that anticoagulant and or blood located in the arterial tubing 72f does not return into the substitution reservoir 76.

The output end of the arterial tubing 72a may be fluidly connected to an arterial input of a primary filter 90. The primary filter 90 is configured to receive blood at the arterial input and separate plasma components of the blood from cellular components of the blood. The primary filter 90 outputs the cellular components of the blood at a cellular components venous output of the primary filter 90 and the plasma components of the blood at a plasma output of the primary filter 90. The cellular components venous output is fluidly connected to the input end of the cellular components venous tubing 72b. Similarly, the plasma output of the primary filter 90 is fluidly connected to the input end of the plasma tubing 72c. That is, the plasma component of the blood is transported through the plasma tubing 72c, while the cellular components of the blood are transported through the cellular components venous tubing 72b.

The pressure of the plasma components of the blood located in the plasma tubing 72c is monitored by the ultrafiltrate pressure sensor 20c. The plasma tubing 72c may include a non-return valve 82 located near the input end of the plasma tubing 72c than the ultrafiltrate pressure sensor 20c. The non-return valve 82 is configured to prevent the contents of the plasma tubing 72c from flowing back into the primary filter 90. The plasma tubing 72c may additionally include and/or pass through a hemoglobin detector 92 located nearer the output end of the plasma tubing 72c than the ultrafiltrate pressure sensor 20c.

The hemoglobin detector is configured to detect hemoglobin in the plasma components of the blood. Detected hemoglobin signifies the presence of red blood cells in the plasma components of the blood. As the red blood cells are typically separated by the primary filter 90 to the cellular components of the blood (i.e. separated from the plasma components of the blood), the presence of hemoglobin signifies a possible issue with the primary filter 90. The hemoglobin detector 92 may be connected to the electrical circuitry in order to pass data regarding the absence or presence of hemoglobin in the plasma tubing 72c. Upon detection of hemoglobin, the electrical circuitry may issue a warning, prevent return of fluids in the tube set 26 to the patient, output the contents of the plasma tubing 72c from the tube set 26, and/or cease operation of the at least one pump 30 of the therapeutic apheresis system 10. The warning may be issued through the display 34 and/or speakers included in the therapeutic apheresis system 10.

In order to output the contents of the plasma tubing 72c if hemoglobin is detected, the tube set 26 may additionally include waste tubing 72g having an input end and an output end. The input end of the waste tubing 72g may be fluidly connected to the plasma tubing 72c nearer the output end of the plasma tubing 72c than the hemoglobin detector 92. The therapeutic apheresis system 10 may include a waste clamp 94 configured to block fluid flow through the waste tubing 72g when activated. The waste clamp 94 may be located between the input end of the waste tubing 72g and the output end of the waste tubing 72g. In this way, if hemoglobin is detected in the plasma tubing 72c, the waste clamp 94 may be opened in order to allow the contents of the plasma tubing 72c to flow through the waste tubing 72g into a waste reservoir 96. The output end of the waste tubing 72g is fluidly connected with the waste reservoir 96. During normal operation (e.g., when hemoglobin is not detected), the waste clamp 94 may be maintained in a closed position such that the contents of the plasma tubing 72c are prevented from flowing through the waste tubing 72g and into the waste reservoir 96.

The plasma components of the blood are transported through the plasma tubing 72c to a column 100. That is, the input of the column 100 is fluidly connected to the output end of the plasma tubing 72c. The column 100 is configured to receive the plasma of the blood at the input of the column 100 and remove unwanted constituents from the plasma of the blood. The filtered plasma of the blood is output at the output of the column 100. The output of the column 100 is fluidly connected with the input end of the post-column tubing 72d.

The column 100 may use any suitable method for removing unwanted constituents from the plasma of the blood. For example, the column 100 may remove unwanted constituents from the plasma of the blood as described in U.S. Pat. No. 8,197,430, filed on Nov. 10, 2000, which is hereby incorporated by reference in its entirety.

The pressure of the filtered plasma components of the blood in the post-column tubing 72d may be monitored by the post-column pressure sensor 20d. The filtered plasma components of the blood may be propelled through the post-column tubing 72d by a plasma pump 30b. That is, the therapeutic apheresis system 10 may include the plasma pump 30b located at a point along the post-column tubing 72 between the post-column pressure sensor 20d and the output end of the post-column tubing 72d. The plasma pump 30b applies force to the post-column tubing 72d such that the contents of the post-column tubing 72d are propelled towards the output end of the post-column tubing 72d.

The post-column tubing 72d may include two markers 78b indicating the position for connecting the plasma pump 30b to the post-column tubing 72d. That is, the post-column tubing 72d may be connected to the therapeutic apheresis system 10 such that the plasma pump 30b interacts with the post-column tubing 72d between the two markers 78b. The markers 78b may also function to maintain the position of the post-column tubing 72d relative to the plasma pump 30b.

The electrical circuitry may also be configured to control the plasma pump 30b such that the pressure measured by the post-column pressure sensor 20d is maintained within a predefined plasma pressure range. The predefined plasma pressure range may be set by and/or adjusted by a user. Alternatively, the predefined pressure range may be a default setting of the system 10.

The post-column tubing 72d may additionally include a particle trap filter 102 located between the post-column pressure sensor 20d and the output of the column 100. The particle trap filter 102 is configured to remove any debris from the plasma components of the blood. Particles, e.g., may be introduced as the plasma moves through the column 100. The particle trap filter 102 may comprise any suitable structure capable of filtering particles from the plasma while allowing the flow of plasma through the particle trap filter 102.

The output end of the post-column tubing 72d may be fluidly connected with a plasma input of a bubble trap filter 106. Similarly, the output end of the cellular components venous tubing 72b may be fluidly connected to the cellular content input of the bubble trap filter 106. That is, the bubble trap filter 106 is configured to receive cellular components of blood at the cellular content input and plasma components of the blood at the plasma input. The bubble trap filter 106 combines the plasma components of the blood and the cellular components of the blood into a blood mixture and outputs the blood mixture at an output of the bubble trap filter 106. Thus, the bubble trap filter 106 outputs filtered blood. The output of the bubble trap filter 106 is fluidly connected to the input end of the venous tubing 72.

The bubble trap filter 106 may comprise a cylinder configured to receive plasma and the cellular contents of blood and allow the two to passively mix before exiting the bubble trap filter 106. The bubble trap filter 106 may be arranged along a gravitational axis such the blood mixture exits the bubble trap filter 106 at a point below the entrance of the plasma and the cellular contents of the blood. In this way, air bubbles within the blood mixture may float to the top of the blood mixture in a direction opposite the output of the bubble trap filter 106. Thus, the presence of air bubbles in the blood mixture may be reduced prior to exiting the bubble trap filter 106.

The bubble trap filter 106 may additionally include a vent line 107 configured to adjust the pressure buildup in the bubble trap filter 106 and/or adjust performance of the bubble trap filter 106.

The filtered blood output by the bubble trap filter 106 enters the venous tubing 72. The venous pressure sensor 20e monitors the pressure of the filtered blood located within the venous tubing 72. The venous tubing 72 is connected to the blood supply of the patient such that the filtered blood is returned to the circulation of the patient.

The venous tubing 72 may also include a bubble detector 108 located between the venous pressure sensor 20e and the output end of the venous tubing 72e. A venous clamp 110 may also be located between the bubble detector 108 and the output end of the venous tubing 72e. The venous clamp 110 is configured to block fluid flow through the venous tubing 72e when activated.

The bubble detector 108 and/or the venous clamp 110 may be operatively connected to the electrical circuitry. That is, the bubble detector 108 may be connected to the electrical circuitry such that the bubble detector 108 is configured to supply data to the electrical circuitry identifying the presence or absence of bubbles in the venous tubing 72e. The venous clamp 110 may be connected to the electrical circuitry such that the electrical circuitry controls operation of the venous clamp 110. For example, if a bubble is detected by the bubble detector 108, the electrical circuitry may control the venous clamp 110 in order to prevent flow of the filtered blood along the venous tubing 72e and into the patient.

The bubble detector 108 may detect the presence of bubbles using light, sound, or any other suitable means for detecting bubbles in a fluid. The venous clamp 110 may comprise any structure suitable for preventing fluid flow through a tube upon activation. For example, the venous clamp 110 may include a solenoid that, upon activation by the electrical circuitry, moves to pinch the venous tubing 72e such that fluid flow through the venous tubing 72e is retarded.

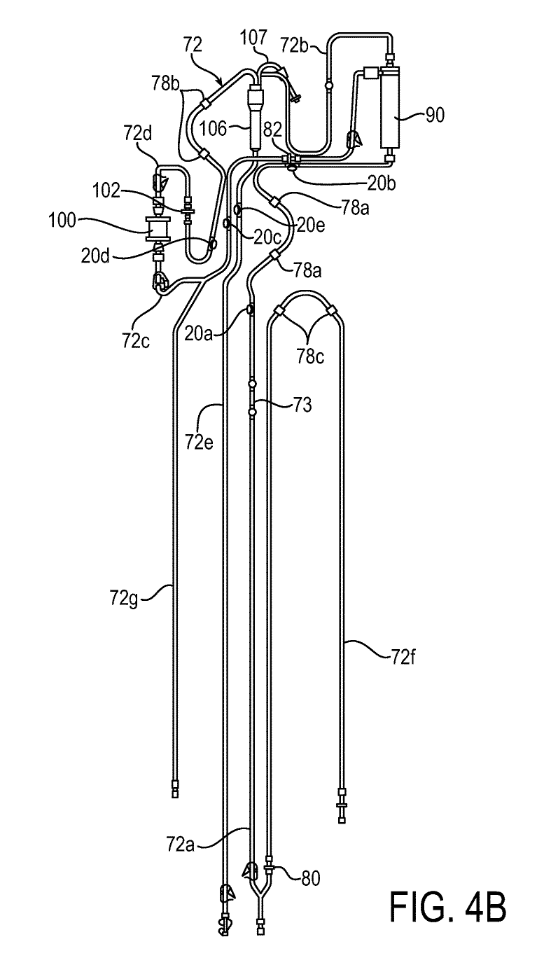

FIG. 4A depicts a tube set 26 disconnected from the therapeutic apheresis system 10. In the depicted embodiment, the bubble trap filter 106 is included as a component of the tube set 26 while the primary filter 90 and the column 100 are not included as components of the tube set 26. Rather, in this example, the primary filter 90 and the column 100 are components of the therapeutic apheresis system 10. In another embodiment, shown in FIG. 4B, the primary filter 90 and the column 100 are included as component of the tube set 26. The tube set 26 may be single use and disposable. That is, the tube set 26 may be single use. By using pressure sensors that are disposable (i.e. single use pressure sensors), errors due to pressure sensor age may be reduced or eliminated.

The different portions of the tube set 26 may be connected together using Luer locks. For example, the arterial tubing 72a, the cellular components venous tubing 72b, and the plasma tubing 72c may be connected to the primary filter 90 via Luer locks. Similarly, the plasma tubing 72c and the Post column tubing 72d may be connected to the column 100 via Luer locks.

The pumps 30 of the therapeutic apheresis system 10, i.e., the arterial pump 30a, the plasma pump 30b, and the substitution pump 30c, may be composed of any suitable pump 30 for applying force to the tubing 72 in order to propel the contents of the tubing 72 in a given direction. For example, the pumps 30 may each be a peristaltic pump. That is, the arterial pump 30a, the plasma pump 30b, and/or the substitution pump 30c may be a peristaltic pump.

Turning to FIG. 1A, the therapeutic apheresis system 10 may include a primary filter holding bracket 112, a column holding bracket 114, and/or a bubble trap filter holding brackets 116. The primary filter holding bracket 112 is configured to hold and maintain the position of the primary filter 90 with respect to the panel 14. Similarly, the column holding bracket 114 is configured to hold and maintain the position of the column 100 relative to the panel 14. The bubble trap filter holding brackets 116 is configured to hold and maintain the position of the bubble trap filter 106 relative to the panel 14.

The primary filter holding bracket 112 may be located on a first side of the therapeutic apheresis system 10. The column holding bracket 114 may be located on a second side of the therapeutic apheresis system 10 opposite the first side. The bubble trap filter holding bracket 116 may be located near a center of the panel 14 between the primary filter holding bracket 112 and the column holding bracket 114. In the depicted embodiment, the primary filter holding bracket 112 holds the primary filter 90 on the right side of the panel 14. In this embodiment, the column holding bracket 114 maintains the position of the column 100 on the left side of the panel 14. The bubble trap filter 106 is held by the bubble trap filter holding bracket 116 near the center of the panel 14 below the display 34. The primary filter 90, column 100, and bubble trap filter 106 are maintained in a position along which the long axis of the primary filter 90, column 100, and bubble trap filter 106 are approximately parallel with gravity. In this embodiment, the arterial pump 30a, plasma pump 30b, and venous pump 30c are located at positions at approximately the edges of the panel 14. The receptacles 74 are positioned on the panel 14 between the pumps 30a-c and holding brackets 112, 114, 116. This configuration, allows a user to accurately connect and position the tube sets 26 to the therapeutic apheresis system 10. The input end of the arterial tubing 72a and the output end of the venous tubing 72e may be directed towards an edge of the panel 14 opposite the display 34 to enable connection of the tube set 26 to a patient without interfering with other portions of the tube set 26.

The covering 12 may be supported by a column 120 attached to a base 122 connected to a movement mechanism 124. The movement mechanism 124 may comprise wheels (e.g., castor wheels). The column 120 and base 122 may be composed of any suitable material for providing support to the covering 12. For example, the column 120 and base 122 may have a metal interior surrounded by an outer plastic covering.

Although the disclosure has been shown and described with respect to a certain embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the disclosure. In addition, while a particular feature of the disclosure may have been described above with respect to only one or more of several illustrated embodiments, such feature may be combined with one or more other features of the other embodiments, as may be desired and advantageous for any given or particular application.

1. A tube set including tubing and at least one in-line pressure sensor, the in-line pressure sensor comprising:
a housing including:
a fluid flow passage;
a window open to the fluid flow passage; and
a rigid plug portion fixed in relation to the housing and extending in a direction transverse to the fluid flow passage, the rigid plug portion including at least one electrical contact for mating with an external component; and
a pressure transducer including a sensing region in pressure communication with the passage and a wiring contact in electrical connection with the at least one electrical contact;
wherein the at least one in-line pressure sensor is fluidly connected with a portion of the tubing.

2. (canceled)

3. An apheresis system comprising:
an in-line pressure sensor comprising:
a housing including:
a fluid flow passage;
a window open to the fluid flow passage; and
a rigid plug portion fixed in relation to the housing and extending in a direction transverse to the fluid flow passage, the rigid plug portion including at least one electrical contact for mating with an external component; and
a pressure transducer including a sensing region in pressure communication with the passage and a wiring contact in electrical connection with the at least one electrical contact;
a panel;
a printed circuit board (PCB) mounted adjacent a back side of the panel and including at least one electrical connector, wherein the panel has an aperture that is aligned with the electrical connector and through which the rigid plug portion of the in-line pressure sensor extends and makes electrical connection with the electrical connector.

4. (canceled)

5. An apheresis system comprising:
a covering having a panel;
at least one peristaltic pump supported on the panel;
a display on the panel;
a primary filter for receiving blood and separating a cellular component of the blood from a plasma component of the blood;
at least one receptacle for receiving a portion of a tube set that connects the pump to the primary filter; and
electrical circuitry contained within the housing to control operation of the at least one peristaltic pump.

6. The apheresis system of claim 3, further comprising a tube set including tubing and the in-line pressure sensor, wherein the at least one in-line pressure sensor is fluidly connected with a portion of the tubing.

7. The tube set of claim 1, wherein:
the tube set includes a plurality of in-line pressure sensors;
the plurality of in-line pressure sensors include an arterial pressure sensor, a pre-filter pressure sensor, an ultrafiltrate pressure sensor, a post-column pressure sensor, and a venous pressure sensor; and
the tubing comprises multiple portions including:
arterial tubing having an input end and an output end and including the arterial pressure sensor and the pre-filter pressure sensor, the arterial pressure sensor located closer to the input end of the arterial tubing than the pre-filter pressure sensor;
cellular components venous tubing having an input end and an output end;
plasma tubing having an input end and an output end and including the ultrafiltrate pressure sensor;
post-column tubing having an input end and an output end and including the post-column pressure sensor; and
venous tubing having an input end and an output end and including the venous pressure sensor.

8. The tube set of claim 7, wherein the tube set further comprises a bubble trap filter having a plasma input, a cellular content input, and an output, wherein:
the bubble trap filter is configured to receive cellular components of blood at the cellular content input, receive a plasma component of the blood at the plasma input, combine the plasma component of the blood and the cellular components of the blood into a blood mixture, and output the blood mixture at the output of the bubble trap filter;
the plasma input of the bubble trap filter is fluidly connected with the output end of the post-column tubing;
the cellular content input of the bubble trap filter is fluidly connected with the output end of the cellular components venous tubing; and
the output of the bubble trap filter is fluidly connected to the input end of the venous tubing.

9. The tube set of claim 7, wherein the tube set further comprises:
a primary filter having an arterial input, a plasma output, and a cellular components venous output, wherein:
the primary filter is configured to receive blood at the arterial input, separate a plasma component of the blood from cellular components of the blood, output the cellular components of the blood at the cellular components venous output of the primary filter, and output the plasma component of the blood at the plasma output of the primary filter;
the arterial input of the primary filter is fluidly connected to the output end of the arterial tubing;
the cellular components venous output of the primary filter is fluidly connected to the input end of the cellular components venous tubing; and
the plasma output of the primary filter is fluidly connected to the input end of the plasma tubing; and
a column having an input and an output, wherein:
the column is configured to receive the plasma of the blood at the input of the column, remove a constituent from the plasma of the blood, and output the plasma of the blood at the output of the column;
the input of the column is fluidly connected with the output end of the plasma tubing; and
the output of the column is fluidly connected with the input end of the post-column tubing.

10. The apheresis system of claim 3, wherein the panel includes a receptacle for receiving a length of the tubing.

11. The apheresis system of claim 3, wherein the panel includes a plurality of receptacles for holding respective portions of the tube set.

12. The apheresis system of claim 10, wherein the receptacle includes a clip or a channel.

13. The apheresis system of claim 5, further comprising a printed circuit board (PCB) mounted adjacent a back side of the panel and including at least one electrical connector, wherein the panel has an aperture that extends from a front side of the panel to a back side of the panel and is aligned with the electrical connector.

14. The apheresis system of claim 3, wherein the aperture includes a retainer for holding the rigid plug portion in engagement with the electrical connector when the plug portion is engaged with the electrical connector.

15. The apheresis system of claim 3, wherein the at least one electrical connector is electrically connected to an amplifier disposed on the PCB.

16. The apheresis system of claim 3, further comprising an arterial pump applying force to the arterial tubing at a location between the arterial pressure sensor and the pre-filter pressure sensor such that the contents of the arterial tubing are propelled towards the output end of the arterial tubing.

17. The apheresis system of claim 16, further comprising:
a substitution pump; and
substitution tubing having an input end and an output end, wherein:
the input end of the substitution tubing is fluidly connected to a substitution reservoir;
the substitution pump applying force to the substitution tubing and propelling the contents of the substitution tubing towards the output end of the substitution tubing; and
the output end of the substitution tubing is fluidly coupled to the arterial tubing nearer the input end of the arterial tubing than the arterial pressure sensor.

18. The apheresis system of claim 17, wherein the substitution tubing includes a substitution non-return valve located nearer the output end of the substitution tubing than the substitution pump.

19. The apheresis system of claim 17, wherein the substitution pump is a peristaltic pump.

20. The apheresis system of claim 16, wherein the arterial pump is a peristaltic pump.

21. The apheresis system of claim 3, further comprising a plasma pump applying force to the post-column tubing at a location between the post-column pressure sensor and the output end of the post-column tubing such that the contents of the post-column tubing are propelled towards the output end of the post-column tubing.

22. The apheresis system of claim 21, wherein the plasma pump is a peristaltic pump.

23. The apheresis system of claim 21, wherein the post-column tubing additionally includes a particle trap filter located between the post-column pressure sensor and the plasma pump.

24. The apheresis system of claim 3, further comprising:
a tube set including tubing and the in-line pressure sensor, wherein:
the at least one in-line pressure sensor is fluidly connected with a portion of the tubing;
the tube set includes a plurality of in-line pressure sensors;
the plurality of in-line pressure sensors include an arterial pressure sensor, a pre-filter pressure sensor, an ultrafiltrate pressure sensor, a post-column pressure sensor, and a venous pressure sensor; and
the tubing comprises multiple portions including:
arterial tubing having an input end and an output end and including the arterial pressure sensor and the pre-filter pressure sensor, the arterial pressure sensor located closer to the input end of the arterial tubing than the pre-filter pressure sensor;
cellular components venous tubing having an input end and an output end;
plasma tubing having an input end and an output end and including the ultrafiltrate pressure sensor;
post-column tubing having an input end and an output end and including the post-column pressure sensor; and
venous tubing having an input end and an output end and including the venous pressure sensor; and
a non-return valve, wherein the non-return valve is located nearer the input end of the plasma tubing than the ultrafiltrate pressure sensor.

25. The apheresis system of claim 3, further comprising:
a tube set including tubing and the in-line pressure sensor, wherein:
the at least one in-line pressure sensor is fluidly connected with a portion of the tubing;
the tube set includes a plurality of in-line pressure sensors;
the plurality of in-line pressure sensors include an arterial pressure sensor, a pre-filter pressure sensor, an ultrafiltrate pressure sensor, a post-column pressure sensor, and a venous pressure sensor; and
the tubing comprises multiple portions including:
arterial tubing having an input end and an output end and including the arterial pressure sensor and the pre-filter pressure sensor, the arterial pressure sensor located closer to the input end of the arterial tubing than the pre-filter pressure sensor;
cellular components venous tubing having an input end and an output end;
plasma tubing having an input end and an output end and including the ultrafiltrate pressure sensor;
post-column tubing having an input end and an output end and including the post-column pressure sensor; and
venous tubing having an input end and an output end and including the venous pressure sensor; and
a hemoglobin detector located nearer the output end of the plasma tubing than the ultrafiltrate pressure sensor.

26. The apheresis system of claim 25, the tube set further comprising waste tubing having an input end, an output end, and a waste clamp, wherein:
the input end of the waste tubing is fluidly connected to the plasma tubing nearer the output end of the plasma tubing than the hemoglobin detector;
the waste clamp is configured to block fluid flow through the waste tubing when activated and the waste clamp is located between the input end of the waste tubing and the output end of the waste tubing; and
the output end of the waste tubing is fluidly connected with a waste reservoir.

27. The apheresis system of claim 3, further comprising:
a tube set including tubing and the in-line pressure sensor, wherein:
the at least one in-line pressure sensor is fluidly connected with a portion of the tubing;
the tube set includes a plurality of in-line pressure sensors;
the plurality of in-line pressure sensors include an arterial pressure sensor, a pre-filter pressure sensor, an ultrafiltrate pressure sensor, a post-column pressure sensor, and a venous pressure sensor; and
the tubing comprises multiple portions including:
arterial tubing having an input end and an output end and including the arterial pressure sensor and the pre-filter pressure sensor, the arterial pressure sensor located closer to the input end of the arterial tubing than the pre-filter pressure sensor;
cellular components venous tubing having an input end and an output end;
plasma tubing having an input end and an output end and including the ultrafiltrate pressure sensor;
post-column tubing having an input end and an output end and including the post-column pressure sensor; and
venous tubing having an input end and an output end and including the venous pressure sensor
the venous tubing additionally includes a bubble detector located between the venous pressure sensor and the output end of the venous tubing.

28. The apheresis system of claim 27, wherein the venous tubing further includes a venous clamp located between the bubble detector and the output end of the venous tubing, the venous clamp configured to block fluid flow through the venous tubing when activated.
